(19) United States
(12) Patent Application Publication
(10) Pub. No.: US 2011/0238091 A1
(43) Pub. Date: Sep. 29, 2011

(54) Hernia Repair With Two-Sided Flexible Prosthesis

(76) Inventor: E. Aubrey Woodroof, Carlsbad, CA (US)

(21) Appl. No.: 12/876,161

(22) Filed: Sep. 5, 2010

Related U.S. Application Data
(60) Provisional application No. 61/318,276, filed on Mar. 26, 2010.

(51) Int. Cl. A61B 17/00

(52) U.S. Cl. 606/151

(57) ABSTRACT

The invention is comprised of a layer of smooth silicone with an embedded layer of nylon weave fixedly attached to one side, the nylon weave impregnated with any of several therapeutic agents, such as hypoallergenic Type I porcine collagen peptide, Immuno-10, chondroitin 4 and 6 sulfate, Vitamin C, and Vitamin E. The invention is optionally rimmed with a bead of silicone on its silicone side, to aid surgeon's in placing the prosthesis over a hernia or other wound site.

### RELATED APPLICATIONS

This application claims the benefit of Provisional Application 61/318,276, filed Mar. 26, 2010.

### FIELD OF INVENTION

The present invention relates to surgically-implantable prostheses that limit the incidence of postoperative adhesions. Specifically, this invention relates to the field of hernia repair prostheses.

### BACKGROUND OF THE INVENTION

Prosthetic mesh materials and the like have been used to reinforce the abdominal wall and to close abdominal wall defects. In almost every case, including incisional and umbilical hernia repair and chest reconstruction, prosthetic mesh structures will come into direct contact with the abdominal viscera. Postoperative adhesions between the mesh and the intestine may occur, potentially leading to intestinal fistulization, and other problems.

The prior art contains several attempts to minimize postoperative adhesions associated with prosthetic mesh materials. Covering the prosthesis with peritoneum or other tissue to form a biological barrier between the implant and the bowel is one approach. Placement of a physical barrier between the surgical site and the surrounding tissue where adhesions are most commonly encountered is another.

U.S. Pat. No. 5,002,551 discloses a physical barrier formed of a knitted oxidized regenerated cellulose (Intercede(TC7)). The patent teaches that other physical barriers can include silicone elastomers and absorbable gelatin films. Based on studies in the literature, such physical barriers alone are not sufficient to reinforce the abdominal wall or to repair abdominal wall defects.

Jenkins et al., “A Comparison of Prosthetic Materials Used to Repair Abdominal Wall Defects”, Surgery, Vol. 94, No. 2, August 1983, pg. 392-398, describes a technique of placing an absorbable gelatin film (Gelfilm®) freely between a piece of Marlex® knitted polypropylene monofilament mesh and the abdominal viscera. The gelatin film dissolved after one week. Thereafter, the incidence of adhesions was reported to be the same as with using the Marlex mesh alone.

Several existing products address this problem.

Cousin Biotech Intramesh® T1, a two-sided polypropylene and ePTFE wall reconstruction prosthesis. Its two-sided structure has the advantage of one smooth side which reduces visceral adherence and a knitted side for better colonization of the peritoneum. One side in polypropylene monofilament knit, the other smooth anti-adhesive ePTFE.

Ethicon (Johnson&Johnson)—ULTRAPRO Partially Absorbable Lightweight Mesh. This was the first partially absorbable mesh available in the United States. They claim strong, secure repair with a flexible scar that lets abdominal wall move more naturally, as well as construction with trusted, proven materials.

### SUMMARY OF THE INVENTION

The present invention is a prosthesis for reinforcing and repairing a weakened muscular that limits the incidence of postoperative adhesions, as well as providing a means for introducing therapeutic materials to the wound site. The invention is formed of a biologically compatible, flexible and non-porous implantable material that reinforces tissue and closes tissue defects, particularly in the abdominal cavity, and a non-porous barrier that physically isolates the reinforcing material from areas likely to form adhesions, such as the abdominal viscera. The barrier and implantable material are permanently attached by a manner which provides a wealth of openings in the material for tissue in-growth.

For repair of abdominal hernia's, it is minimally desired that there be one surface facing the gut that is non-adhesive and a surface facing away from the gut that permits tissue ingrowth, can become vascularized, and is indefinitely stable.

In the preferred embodiment of the invention, the invention is comprised of attached sheets of knitted nylon monofilament mesh fabric and a silicone elastomer. The nylon is 12/1 (0.006″) or 15/2 (0.010″) denier. 15/1 nylon has also been used. The silicone is a non-porous membrane approximately 0.001″ thick. An optional seam is stitched around the edge of the membrane to provide strength and control elongation of the invention as applied to a rupture. An alternate embodiment of the invention is to have an extra bead of RTV silicone applied around the edge of the prosthesis, which would be molded in during the layering process. Because of its composition, the invention is light, translucent, and very flexible.

In another alternate embodiment of the invention, the invention can be used for vascular repair, e.g. re-enforcement and protection of aneurisms from blow out, protection of anastomosis after vascular repair, and repair/replacement of portions of necrotic cardiac muscle tissue when combined with cardiomyocytes.

Addition of hypoallergenic Type I porcine collagen peptide or other biologicals (Immuno-10, chondroitin4 and 6 sulfate, vitamin C and E) is possible for therapeutic purposes. These materials would be added to the mesh side of the invention.

The synthetic components of the invention are stable in vivo and resistant to mammalian enzymes.

It is an object of the invention to provide a prosthesis which combines the attributes of a surgical mesh fabric and of a physical barrier.

It is a further object of the invention to provide a prosthesis which stimulates tissue infiltration without inflammation of the abdominal viscera.

It is a further object of the invention that the prosthesis can be sterilized with E-beam irradiation and will have an expected three-year shelf life at room temperature.

It is a further object of the invention that it be thin,
strong (hold sutures, staples and the like), transparent, very flexible; stretchable w/ability to contract, stable indefinitely in biological systems (in vivo humans),
non-toxic,
hypoallergenic, and
non-pyrogenic.

### BRIEF DESCRIPTION OF THE DRAWINGS

FIG. 1. Top view of the invention

FIG. 2. Side view of the invention

FIG. 3. Cross-section of the invention

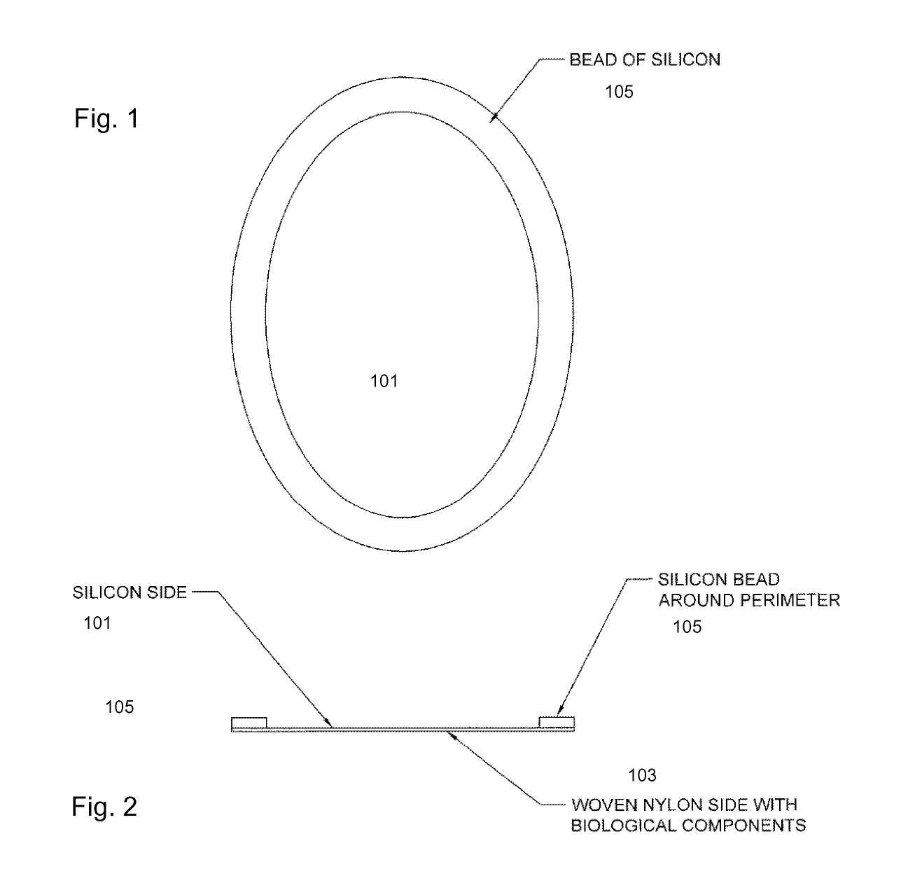

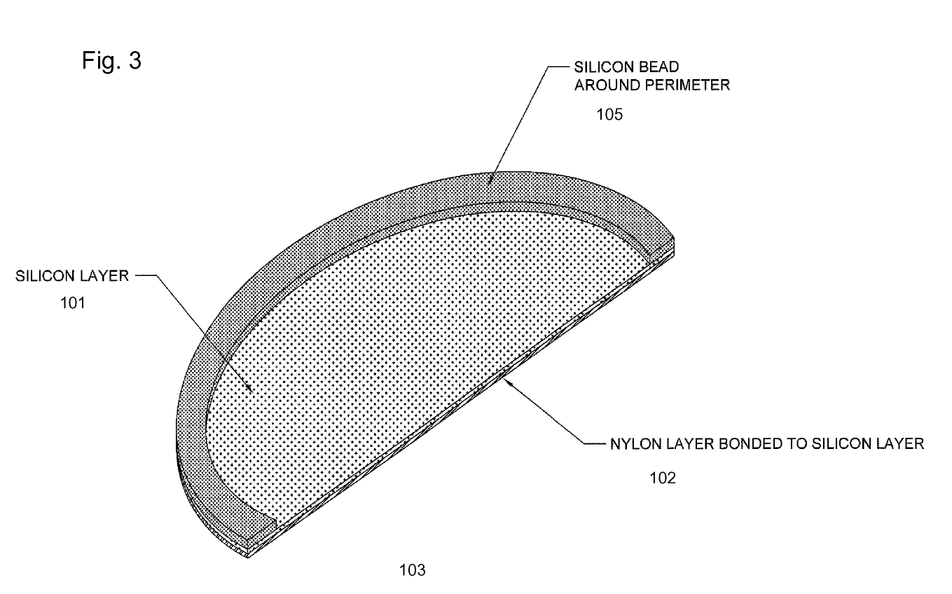

### DETAILED DESCRIPTIONS

The invention 100 is comprised of two sheets of material, a smooth silicone sheet 101 and a woven sheet 102. The silicone sheet 101 and the woven sheet 102 are brought together at time of manufacture by implanting the woven sheet 102 into the silicone sheet 101 while said silicone sheet 101 is still soft. A portion of the woven sheet 102 implants itself in the material of the silicone sheet 101 leaving a thickness of woven material 103 above the level of the silicone sheet 101.

The woven sheet 102 is comprised of nylon thread in the preferred embodiment. The nylon thread can be in several deniers, typically 12/1, 15/1 or 15/2. The preferred embodiment uses 15/1 denier nylon thread in the woven sheet 101.

The woven material 103 above the silicone sheet 101, the “woven side” of the invention, can be impregnated with biological and non-biological substances for therapeutic purposes. These substances include but are not limited to hypoallergenic Type I porcine collagen peptide, Immuno-10, chondroitin 4 and 6 sulfate, Vitamin C, and Vitamin E.

In the preferred embodiment, there is an additional bead of silicone 105 that is added to the “smooth side” of the invention 100, on the surface of the silicone sheet 101, around the edge of the invention 100. This bead of silicone assists the surgeon implanting the device by giving the surgeon something to grip with a forceps. Without the bead of silicone 105, the edge of the invention 100 could slip out of a forceps during implantation.

It should be understood that the foregoing description of the invention is intended merely to be illustrative thereof and that other equivalents, embodiments and modifications of the invention may be apparent to those skilled in the art.

What is claimed is:

1. A flexible prosthesis, the prosthesis comprised of two sheets of material, a smooth silicone sheet and a woven sheet,
the silicone sheet, the “smooth side”, comprised of smooth silicone,
The woven sheet comprised of nylon thread, nylon thread in a plurality of deniers,
the silicone sheet and the woven sheet brought together at time of manufacture by implanting the woven sheet into the silicone sheet while said silicone sheet is still soft,
a portion of the woven sheet implanted in the material of the silicone sheet leaving a thickness of the woven sheet above the level of the silicone sheet,
the woven above the silicone sheet, the “woven side” of the invention, optionally impregnated with biological and non-biological substances for therapeutic purposes.

2. The prosthesis of claim 1 where there is an additional bead of silicone added to the “smooth side” of the invention on the surface of the silicone sheet around the edge of the invention 100, the purpose of this bead of silicone to assists a surgeon implanting the invention by giving the surgeon something to grip with a forceps.

3. The prosthesis of claim 1 where the biological and non-biological substances are selected from the list of hypoallergenic Type I porcine collagen peptide, Immuno-10, chondroitin 4 and 6 sulfate, Vitamin C, and Vitamin E.
